(19) United States
(12) Patent Application Publication
(10) Pub. No.: US 2007/0299520 A1
(43) Pub. Date: Dec. 27, 2007

(54) Surface treatment of implantable devices

(75) Inventors: Hai H. Trieu, Cordova, TN (US); Jeffrey H. Nycz, Collierville, TN (US); Michael C. Sherman, Memphis, TN (US); Jon C. Serbousek, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/474,912

(22) Filed: Jun. 26, 2006

(51) Int. Cl. A61F 2/28

(52) U.S. Cl. 623/16.11; 623/23.6; 623/901

(57) ABSTRACT

A device includes a sterilizing system configured to sterilize an implantable device and includes a coating system configured to apply an osteal functional coating to the implantable device.

### FIELD OF THE DISCLOSURE

This disclosure generally relates to surface treatment of implantable devices.

### BACKGROUND

In human anatomy, skeletal structures can break, joints can degrade over time or in response to excessive strain, and diseases or syndromes can cause deformations in skeletal structures. For example, a bone, such as a tibia or a fibula of the leg or an ulna or a radius in the arm can break when exposed to excessive stress. Similarly, cranial structures and vertebra can break in response to catastrophic forces. In another example, long term exposure to stress and strain can lead to degradation of a joint, such as an intervertebral disc, a knee or an elbow joint, an acromioclavicular (AC) joint, or a glenohumeral joint. In a further example, a disease or syndrome, such as arthritis or osteoporosis, can lead to degradation or deformation of an osteal structure or tissue forming a joint.

Frequently, medical professionals use implantable devices to repair or replace injured or degraded osteal structures or joints. For example, an intervertebral disc can be replaced with a prosthetic disc implant. In another example, a knee joint can be partially or completely replaced with implantable devices. For broken bones, a surgeon can select a device or structure to encourage bone growth or support the bone while the bone is healing.

Typically, an implantable device includes a surface that contacts an osteal structure. The surface configured to contact the osteal structure can be configured to adhere to the osteal structure. Often, the osteal structure desirably grows to further bond with the surface of the implantable device. In another example, the implantable device is configured to degrade or to be absorbed as bone grows. For example, the implantable device can form a structure or matrix on to which bone can grow. In a further example, the implantable device can provide structural support as bone grows to replace a lost or broken bone.

In addition, the implantable device can include a surface that contacts another device or soft tissue. Such surfaces desirably remain free of bone growth. For example, a surface configured to act as a movable surface of a joint can degrade as a result of boney structure formation. In another example, an implantable device configured to act as a degradable structural support can desirably remain free of boney formations.

To effect bone growth or to prevent bone growth, a surface can be treated with active agents. For example, an active agent can be used to induce bone growth or to provide a structure that guides bone growth. Alternatively, an active agent can be used to prevent bone growth on a surface.

In another example, bone growth can be encouraged by the texture of a surface. Nano-sized or micro-sized features on a surface can influence adhesion and anchoring of bone to an implantable device. As such, a surface can be roughened or smoothed to encourage or discourage adhesion to osteal structures.

While surface treatment can be performed as part of the manufacturing process, such treatments can degrade over time and lose effectiveness prior to implantation. For example, active agents can degrade with time or in response to environment. Shipping conditions are difficult to manage and a high temperature imposed during shipping can reduce the activity of an active agent. In another example, movement and vibrations resulting from transportation of devices can delaminate coatings. As such, coatings can flake or fall off of a surface. In a further example, implantable devices can be contaminated over time, such as with dirt or bacteria. In particular, roughened surfaces can form recesses to which bacteria more easily attach or dirt more easily adheres.

### BRIEF DESCRIPTION OF THE DRAWINGS

The present disclosure may be better understood, and its numerous features and advantages made apparent to those skilled in the art by referencing the accompanying drawings.

FIG. 1 and FIG. 2 include illustrations of exemplary treatment devices.

FIG. 3, FIG. 4, and FIG. 5 include illustrations of exemplary implantable devices.

FIG. 6 and FIG. 7 include illustrations of exemplary cartridges.

FIG. 8 and FIG. 9 include illustrations of a surface of an exemplary implantable device.

FIG. 10, FIG. 11, and FIG. 12 include illustrations of exemplary treatment mechanisms.

FIG. 13, FIG. 14, FIG. 15, and FIG. 16 include flow diagrams illustrating exemplary methods associated with devices for treating implantable devices.

FIG. 17 includes an illustration of an exemplary treatment setting.

### DESCRIPTION OF THE EMBODIMENTS

In a particular embodiment, a treatment device can be adapted to receive an implantable device. For example, the treatment device can include a coating system to provide a coating having osteal functionality. In addition, the treatment device can include a sterilization system. In another example, the treatment device includes a texturing system to adapt the texture of a surface for osteal functionality. The treatment device can be detachably coupled to a cartridge including a reservoir configured to store an osteal functional formulation. In particular, the treatment device can be located at a surgical or clinical facility.

In another exemplary embodiment, a method to treat an implantable device can include sterilizing the implantable device and coating the implantable device with an osteal functional coating. In an example, the method can be performed in a clinical setting or at a surgical facility prior to implantation of an implantable device into a patient.

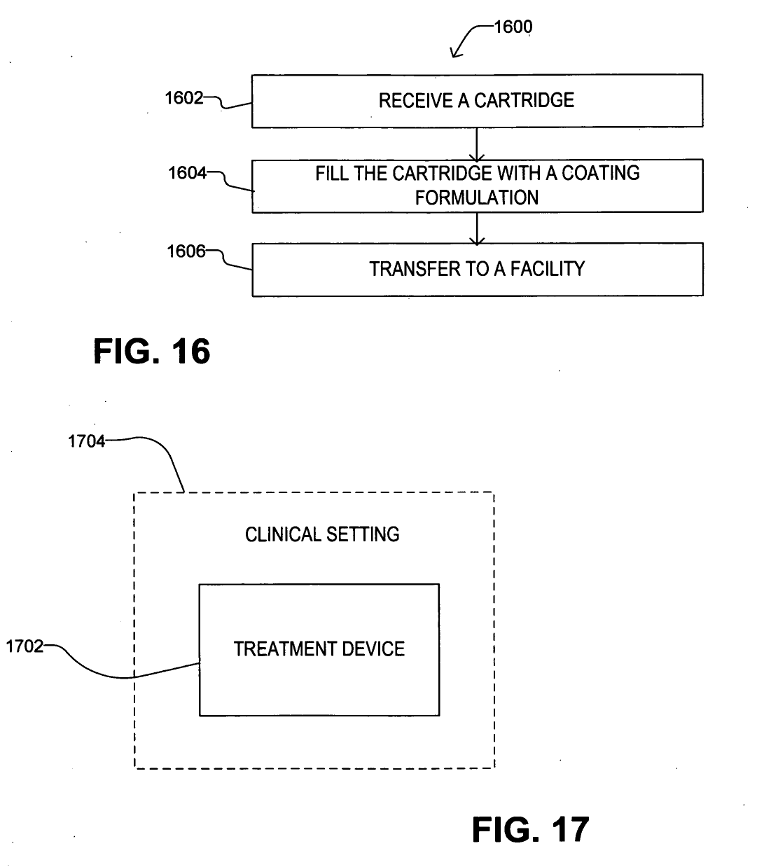

In a further exemplary embodiment, a treatments device can be used to treat an implantable device in a clinical setting, as illustrated at FIG. 17. For example, the treatment device 1702 can be used to surface treat, clean, sterilize, or coat the implantable device, or any combination thereof, to influence the osteal functionality of the implantable device. In particular, the treatment device 1702 can be located at a clinical setting 1704. For example, the clinical setting 1704 can be an operating room, a surgical facility, an outpatient facility, or a hospital. Alternatively, the treatment device 1702 may be located at a facility in the field or after the implantable device leaves the manufacturer.

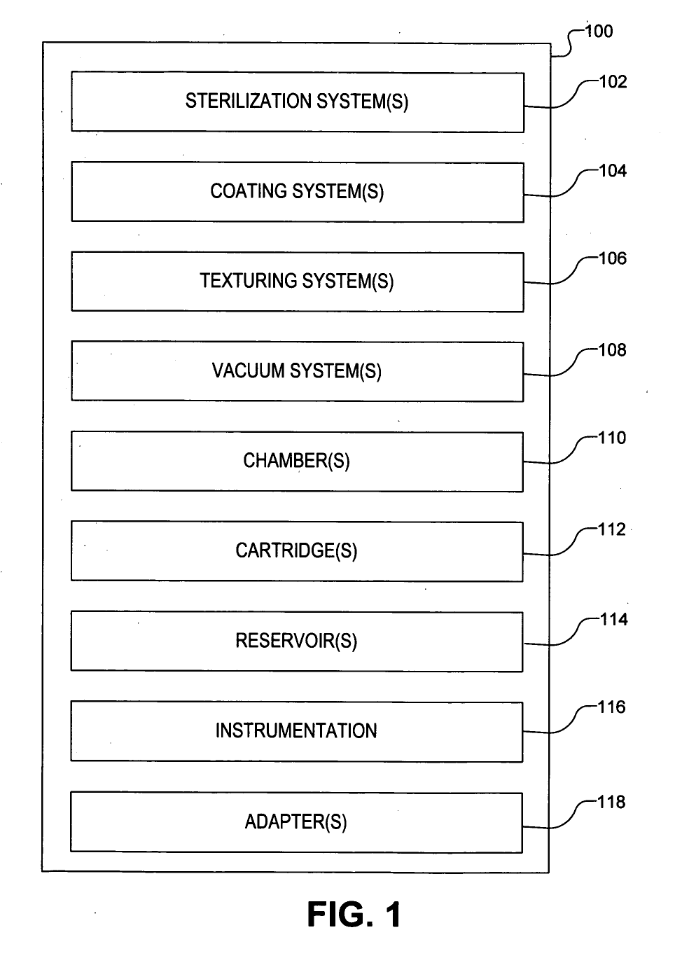

In an exemplary embodiment, FIG. 1 includes an illustration of an exemplary treatment device 100 adapted to treat an implantable device in a manner that influences its osteal functionality. The osteal functionality of an implantable device relates to the effect the implantable device or a surface thereof has on bone growth. A positive osteal functionality, for example, can encourage the formation of new bone (“osteogenesis”), such as through inducing bone growth (“osteoinductivity”) or by providing a structure onto which bone can growth (“osteoconductivity”). Generally, osteoconductivity refers to an implantable device or a surface or portion thereof supporting the attachment of new osteoblasts and osteoprogenitor cells. As such, the implantable device provides an interconnected structure through which new cells can migrate and new vessels can form. Osteoinductivity typically refers to the ability of the implantable device or a surface or a portion thereof to induce nondifferentiated stem cells or osteoprogenitor cells to differentiate into osteoblasts. In another example, a negative osteal functionality can discourage bone growth.

As illustrated, the treatment device 100 can include a sterilization system 102 and a coating system 104. The treatment device 100 further can include a texturing system 106, a vacuum system 108, or a chamber 110. In an example, the treatment device 100 includes a cartridge 112 or a reservoir 114 to store a formulation. Further, the treatment device 100 can include instrumentation 116.

The sterilization system 102 can be configured to sterilize the implantable device. For example, the sterilization system 102 can sterilize through heat, a chemical agent, or radiation. In an example, the sterilization system 102 includes a heater configured to heat a source of water or the chamber 110 of the treatment device 100. As such, the sterilization system 102 can act to autoclave the implantable device or to steam sterilize the implantable device. In such an example, the sterilization system 102 can include a reservoir of demineralized water or distilled water. In particular, the sterilization system 102 can be an autoclave system or a steam injection system. In another example, the sterilization system 102 can be adapted to apply a chemical agent, such as an antiseptic or an antibacterial agent, to the implantable device. For example, the sterilization system 102 can be configured with a nozzle to spray an antiseptic solution onto an implantable device. Alternatively, the sterilization system 102 can be configured to provide an antiseptic solution into which the implantable device is immersed. An exemplary antiseptic solution includes an alcohol solution or a solution including a biocide. In a further example, the sterilization system 102 can include a radiation source. For example, the radiation source can include an ultraviolet electromagnetic radiation source. In another example, the radiation source can include a gamma-radiation source or an x-ray source.

The coating system 104 can be configured to alter the osteal functionality of a surface of an implantable device. For example, the coating system 104 can operate to provide a coating having osteal functionality on the implantable device. In an example, the coating system 104 can spray a coating, deposit a coating or provide a coating formulation into which the implantable device can be dipped.

In an exemplary embodiment, the coating system 104 can coat the implantable device with an osteal functional coating. For example, the osteal functional coating can influence bone growth or formation in proximity to the coating. In an example, the osteal functional coating can be a positive osteal functional coating encouraging bone formation, such as through osteoconductivity or osteoinductivity. Alternatively, the osteal functional coating can be a negative osteal functional coating, discouraging bone growth in proximity to the coating.

An exemplary osteal functional coating can include an active agent. The active agent can be, for example, an osteogenerative agent. For example, an osteogenerative agent can be an osteoinductive agent, an osteoconductive agent, or any combination thereof.

In an example, an osteoconductive agent can provide a favorable scaffolding for vascular ingress, cellular infiltration and attachment, cartilage formation, calcified tissue deposition, or any combination thereof. An exemplary osteoconductive agent includes collagen; a calcium phosphate, such as hydroxyapatite, tricalcium phosphate, or fluorapatite; demineralized bone matrix; or any combination thereof.

In another example, an osteoinductive agent can include bone morphogenetic proteins (BMP, e.g., rhBMP-2); demineralized bone matrix; transforming growth factors (TGF, e.g., TGF-β); osteoblast cells, growth and differentiation factor (GDF), or any combination thereof. In a further example, an osteoinductive agent can include HMG-CoA reductase inhibitors, such as a member of the statin family, such as lovastatin, simvastatin, pravastatin, fluvastatin, atorvastatin, cerivastatin, mevastatin, pharmaceutically acceptable salts esters or lactones thereof, or any combination thereof. With regard to lovastatin, the substance can be either the acid form or the lactone form or a combination of both. In addition, osteoconductive and osteoinductive properties can be provided by bone marrow, blood plasma, or morselized bone of the patient, or other commercially available materials.

In another exemplary embodiment, a positive osteal functional coating can be derived from a formulation including the active agent. For example, the formulation can include a solvent. In another example, the formulation includes a matrix-forming component, such as a dissolved polymer or a polymer precursor. In a particular example, a positive osteal functional coating formulation can include a slurry including osteoconductive particulate and polymer forming constituents. The slurry further can include an osteoinductive active agent.

In a particular embodiment, a coating formulation includes a matrix-forming component, such as a polymer component. A polymer component can include a dissolved polymer or a component that reacts to form a polymer. In an example, the polymer is non-bioresorbable and, when part of a coating, is fixed to the surface indefinitely. In another example, the polymer is bioresorbable.

An exemplary polymer includes polyethylene (PE), polypropylene (PP), polyethylenerephthalate (PET), polyglactine, polyamide (PA), polymethylmethacrylate (PMMA), polyhydroxymethylmethacrylate (PHEMA), polyvinylchloride (PVC), polyvinylalcohole (PVA), polytetrafluorethylene (PTFE), polyetheretherketone (PEEK), polysulfon (PSU), polyvinylpyrolidone, polyurethane, polysiloxane, or any combination thereof. Such a polymer is generally biocompatible.

In another example, the polymer is selected from poly(α-hydroxy acids), poly (ortho esters), poly(anhydrides), poly(amino acids), polyglycolide (PGA), polylactide (PLLA), poly(D,L-lactide) (PDLLA), poly(D,L-lactide co-glycolide) PLGA), poly(3-hydroxybutyricacid) (P(3-HB)), poly(3-hydroxy valeric acid) (P(3-HV)), poly(p-dioxanone) (PDS), poly(ε-caprolactone) (PCL), polyanhydride (PA), copolyetherester, or any combination thereof. Such a polymer is typically biocompatible and bioresorbable. In a further example, an exemplary polymer can include a natural polymer, such as collagen, polypeptide, gelatin, or any combination thereof.

In an alternative embodiment, the coating can be a negative osteal functional coating. An negative osteal functional coating, for example, can encourage soft tissue growth, discourage bone growth, or limit bone attachment to a surface.

In an example, the coating system 104 can be a spray coating system. For example, a spray coating system can include a fluid nozzle configured to spray a coating formulation on a surface of an implantable device. In another example, the spray coating system can include a pump to motivate the coating formulation through the fluid nozzle.

In a further example, the coating system 104 can be a dip coating system. For example, the coating system 104 can cause a surface or portion of an implantable device to contact a coating formulation, such as through moving the implantable device into the coating formulation or by moving a reservoir of the coating formulation to the implantable device. In an example, the coating formulation can be dried to provide a viscous or solid coating.

In a further example, the coating system 104 can be a deposition system, such as a system that deposits ions, atoms, or small molecules on a surface. For example, the coating system 104 can be a plasma deposition system or a vapor deposition system.

The texturing system 106 can adapt a surface texture of an implantable device to influence adhesion of a coating or of osteal structures. For example, roughened surfaces having nano-sized or micro-sized defects can exhibit improved bone adhesion. In an exemplary embodiment, the texturing system 106 can roughen a surface through abrasion. For example, the texturing system 106 can abrade the surface using an abrasive formulation, such as an abrasive slurry or an abrasive powder. In an example, the texturing system 106 can spray or blow the abrasive formulation on the surface. For example, the texture system can include a pneumatic system configured to direct high velocity particulate at the surface of the implantable device. In another example, the surface of the implantable device can be contacted with an abrasive formulation undergoing high shear mixing. In another exemplary embodiment, the texturing system 106 can roughen a surface through the impact of particles or ball bearings. For example, ball bearings can be directed to the surface at high velocity to cause impact dents in the surface of the implantable device. In an additional example, the texturing system 106 can include a chemical etching system and as such, a texturing formulation can include a chemical etchant.

In a further exemplary embodiment, the texturing system 106 and the coating system 104 can be incorporated into a single system. For example, electrical methods can be used to remove material from a surface, such as a metal surface, and can be used to deposit material on the surface, such as through electroplating. In an additional example, a system can be used to induce corrosion, effecting a texturing of the surface by establishing a layer of corroded material. In another example, a printing system can deposit a layer in a pattern, resulting in a coating that both imparts a texture to the surface and influences bone growth.

In addition, the treatment device 100 can include a vacuum system 108. For example, the vacuum system 108 can be used to reduce the pressure in the treatment device 100 to effect removal of a solvent. In an example, solvent can be removed from a coating formulation to form a coating on a surface of the implantable device. In a particular example, the vacuum system 108 can be used to dry the implantable device and equipment between process steps, such as sterilizing and coating.

Further, the treatment device 100 can include one or more chambers 110 in which an implantable device can be placed for treatment. In a particular example, the treatment device 100 includes a single chamber in which sterilization and coating can be performed. The chamber 110 can be configured for vacuum pressures when a vacuum system 108 or a connector to a vacuum system is provided. In another example, the chamber 110 can be configured for pressure higher than atmospheric pressure, such as autoclave pressures.

The treatment device 100 can also include an adapter 118 configured to receive an implantable device. In an example, the adapter 118 can be removed from the chamber 110. In another example, the adapter 118 can be adjustable or can be interchangeable, such as, for example, exchangeable for a second adapter to permit treatment of implantable devices having different designs and configurations.

In addition, the treatment device 100 can include locations for storing treatment formulations, such as coating formulations, texturing formulations, and sterilizing formulations. For example, the treatment device 100 can include one or more reservoirs 114 to store formulations. In a further example, the treatment device 100 can detachably attach to one or more cartridges 112. A cartridge 112 can be configured to store one or more formulations for use with the treatment device 100. For example, a cartridge 112 can be configured to store a coating formulation. In another example, the cartridge 112 can be configured to store a texturing formulation or a sterilizing formulation. In particular, a cartridge 112 can be exchanged to change the functionality of a device, such as through changing the coating formulation.

Further, the treatment device 100 can include instrumentation 116. The instrumentation can include an interface to provide instructions to the treatment device 100. In addition, the instrumentation 116 can include control circuitry, computational circuitry, and memory. In a further embodiment, the treatment device 100 can include an interface to an external computer or a network. For example, the treatment device 100 can be programmed via an external computer or can acquire instructions or protocols from devices accessible via the network.

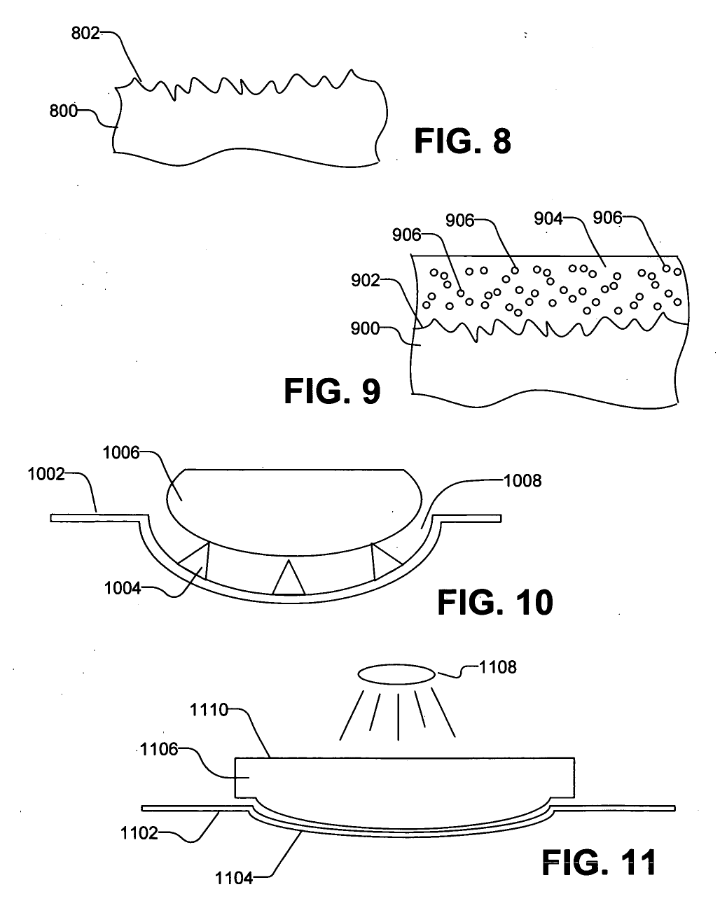

In a particular example, the treatment device 100 can sterilize the surface and form a coating on the surface. In addition, the treatment device 100 can texture a surface. For example, FIG. 8 includes an illustration of a textured surface 802 of an implantable device 800. The surface 802, for example, can be textured by abrasive processes or impact processes. In an example, the surface 802 can include small abrasions that encourage bone adhesion or adhesion of a coating. In another example, the surface 802 can include indentations caused by particle impact.

A coating can be formed over a textured surface. For example, FIG. 9 includes an illustration of a treated implantable device 900. The implantable device 900 can include a textured surface 902 and a coating 904. In an example, the coating 904 includes a matrix material and an active agent. In a particular example, the coating 904 includes particulate material 906, such as demineralized bone matrix, calcium phosphate particles, hydroxyapatite, or any combination thereof. In a further example, the matrix material can include a polymer, such as a biocompatible polymer. For example, the matrix material can include a hydrogel material or a bioresorbable material.

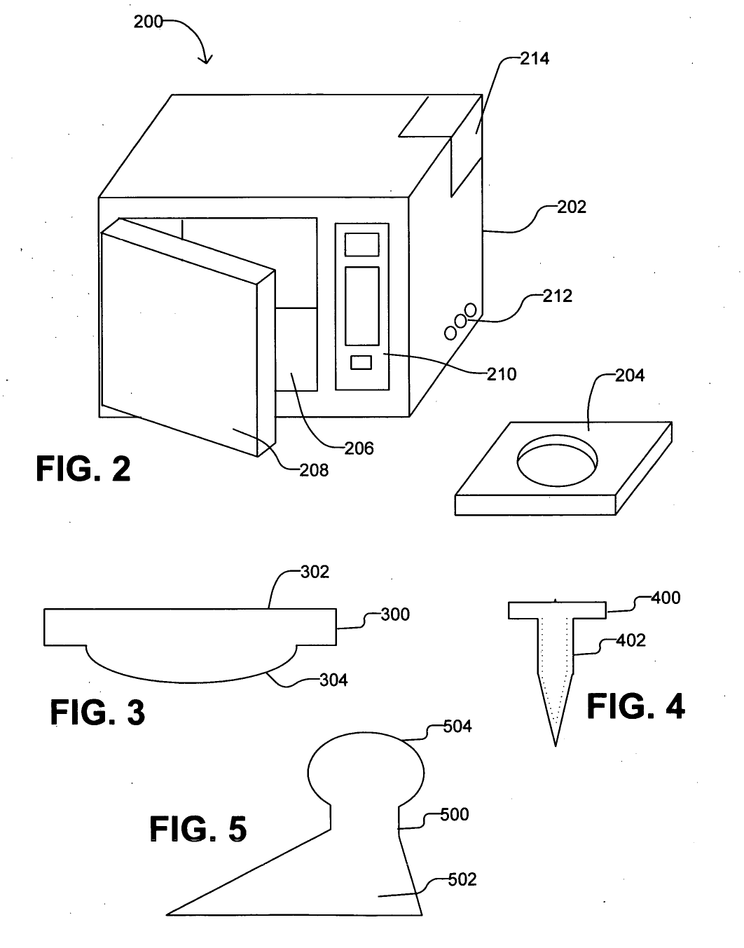

Once treated, the implantable device can include one or more surfaces having osteal functionality. For example, an implantable device can include a positive osteal functional coating. In another example, the implantable device can include a treated region having a positive osteal functionality and a second region that is untreated or has a negative osteal functionality. In a particular example, FIG. 3 includes an illustration of an implantable device 300, such as a part of a prosthetic intervertebral disc. The device 300 can include a surface 302 having a positive osteal functionality configured to encourage vertebral bone adhesion to the surface 302. In addition, the device 300 includes a surface 304 configured to move relative to other components. The surface 304 can be untreated or can be treated to prevent bone growth.

In another example, a device 400, such as a bone screw, can include a surface 402 that has a positive osteal functionality, as illustrated in FIG. 4. For example, bone growth and bone attachment can be encouraged on the surface 402 to prevent movement and loosening of the device 400.

In a further example, a device 500, such as a portion of a prosthetic hip, can include treated regions and untreated regions, as illustrated in FIG. 5. For example, region 502 can be treated to encourage bone growth once implanted in proximity to a bone and region 504 can be untreated.

In addition to the illustrated examples, the treatment device can be used to treat soft tissue implantable devices or hard tissue implantable devices. For example, the treatment device can be configured to treat prosthetic devices to repair knees, hips, shoulders, spinal discs, or teeth. Further, the treatment device can be used to treat devices for anchoring bone, such as dental anchors, or can be used to treat non-union fractures or mal-union fractures.

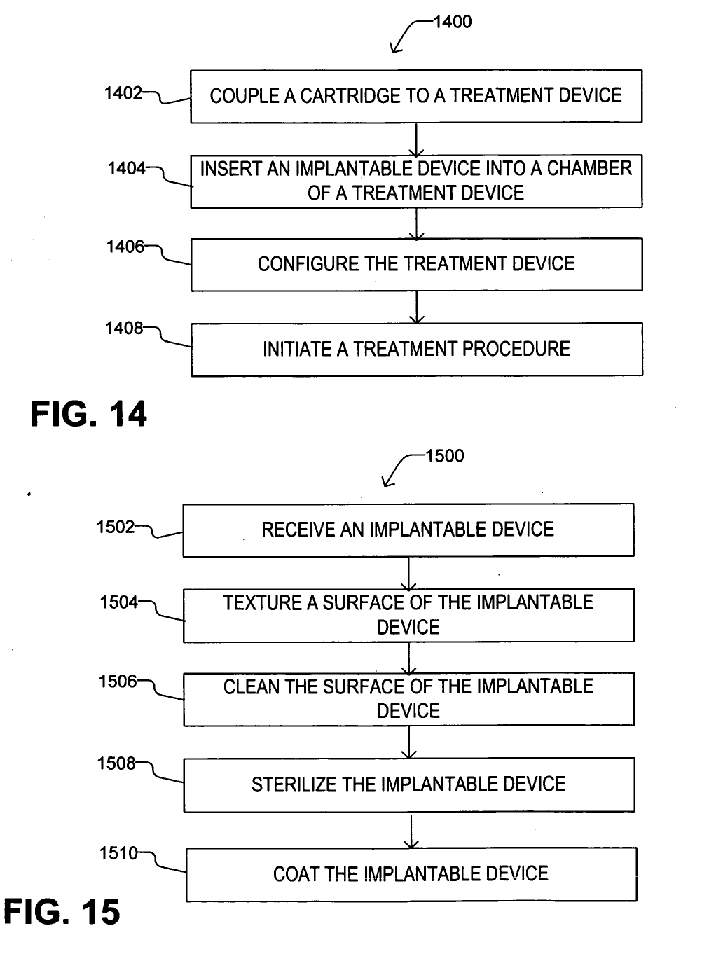

In particular, the treatment device can be used in a clinical setting. For example, an implantable device can be prepared at a surgical facility, as illustrated at 1302 of method 1300 of FIG. 13. In particular, the surgical facility can selectively configure an implantable device based on a desired treatment for a patient. Once treated, the implantable device can be implanted into the patient, as illustrated at 1304.

FIG. 14 includes a flow diagram of an exemplary method 1400 to treat the implantable device. For example, a cartridge including a desired treatment formulation can be coupled to a treatment device, as illustrated at 1402. For example, the cartridge can include a coating formulation that includes active agents prescribed by a physician.

For treatment, the implantable device can be inserted into a chamber of the treatment device, as illustrated at 1404. For example, the implantable device can be inserted into an adapter that is inserted into the chamber.

The treatment device can be configured, as illustrated at 1406. For example, a treatment protocol can be provided to the treatment device via an instrumentation interface. In another example, the treatment protocol can be provided via a connection to an external computer. In a particular example, the external computer includes software providing a graphical user interface that permits entry of treatment parameters. A treatment parameter can include, for example, a length of a treatment, a type of coating, a temperature at which a treatment is to occur, a type or size of an implantable device, a selection or an order of process steps, or any combination thereof. Once the treatment device is configured, the treatment can be initiated, as illustrated at 1408.

Treatment of an implantable device can include texturing a surface of the implantable device, coating a surface of the implantable device, sterilizing the implantable device, or any combination thereof. As illustrated in FIG. 15, a method 1500 to treat an implantable device can include receiving an implantable device in a treatment device, as illustrated at 1502. For example, the implantable device can be placed in an adapter and inserted into the treatment device. An adapter can be selected based on the type of implantable device to be treated and based on what treatment is to be performed.

Optionally, a surface of the implantable device can be textured, as illustrated at 1504. For example, an abrasive formulation can abrade the surface. In another example, a high velocity particulate can impact the surface. In addition, the surface of the implantable device can be cleaned, as illustrated at 1506. For example, abraded material or abrasive particulate can be cleaned from the surface, such as with a cleaning solution or water.

In addition, the implantable device can be sterilized, as illustrated at 1508. For example, the implantable device can be immersed or sprayed with a sterilizing formulation, such as an antiseptic solution. In another example, the implantable device can be irradiated. In a further example, the implantable device can be autoclaved or sterilized with steam.

Further, the implantable device can be coated with an osteal functional coating, as illustrated at 1510. For example, the implantable device can be immersed or sprayed with a coating formulation that forms an osteal functional coating. In another example, the implantable device can be treated with vapor deposition, plasma deposition, or electroplating.

While sterilizing is depicted as occurring before coating, the implantable device alternatively can be sterilized after coating. Further, the implantable device can be textured after coating. For example, a coated surface can be textured to improve bone adhesion.

In a particular example, FIG. 2 includes an illustration of an exemplary treatment device 200. Such a treatment device 200 can be located in a clinical setting and in particular, can be located in a surgical setting. The treatment device 200 can include a housing 202 surrounding a chamber 206 and instrumentation 210. The treatment device 200 can include a door 208 to provide access to the chamber 206. In particular, the door 208 and housing 202 can engage each other to form a chamber 206 capable of pressures exceeding atmospheric pressures, such as autoclave pressures, or capable of pressures below atmospheric pressure, such as pressures useful in lyophilizing or freeze drying processes.

As illustrated, the treatment device 200 can include an instrumentation panel 210. The instrumentation panel 210 can include a display and keys that form a user interface. In a further example, the treatment device 200 can include ports 212 to provide access to external computational devices.

In a further example, the treatment device 200 can be configured to engage a cartridge 214. The cartridge 214 can include a formulation for use during operation of the treatment device 200.

In addition, the treatment device 200 can include a detachable adapter 204. For example, the adapter 204 can be configured to engage a specific type of implantable device. The adapter 204 can engage the implantable device in a manner that permits a desired treatment. Optionally, the adapter 204 can be reconfigured to receive a different type of device. In another example, the adapter 204 can be exchanged for a second adapter configured to receive a second type of implantable device or configured to receive the first type of implantable device to effect a different treatment.

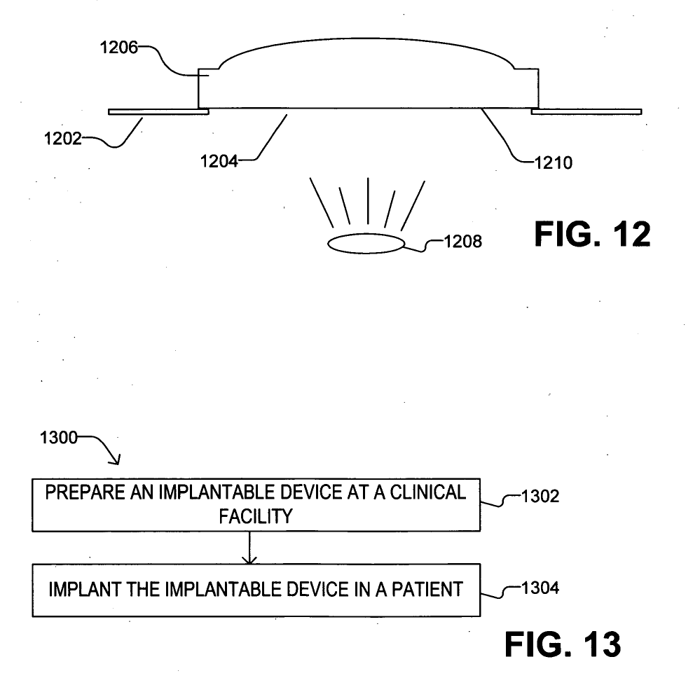

For example, FIG. 10 includes an illustration of an adapter 1002. The adapter 1002 can include support structures 1004 to engage an implantable device 1006. In an example, a space 1008 is formed in which formulations, such as coating formulations can be placed. In a particular example, the space 1008 can be at least partially filed with a coating formulation having a solvent. The solvent can be extracted, such as through evaporation, leaving a coating on a surface of the implantable device 1006.

In another example, FIG. 11 includes an illustration of an adapter 1102 configured to engage an implantable device 1106 in an indentation 1104. An upper surface 1110 can be exposed for treatment. For example, the surface 1110 can be sprayed via a nozzle 1108 with a coating formulation, a sterilizing formulation, a texturing formulation, or any combination thereof.

In a further example, FIG. 12 includes an illustration of an adapter 1202 including an opening 1204. A surface 1210 can be exposed through the opening 1204 and treated, such as by using spray nozzle 1208. In another example, vapor deposition techniques can be used to affect the surface 1210.

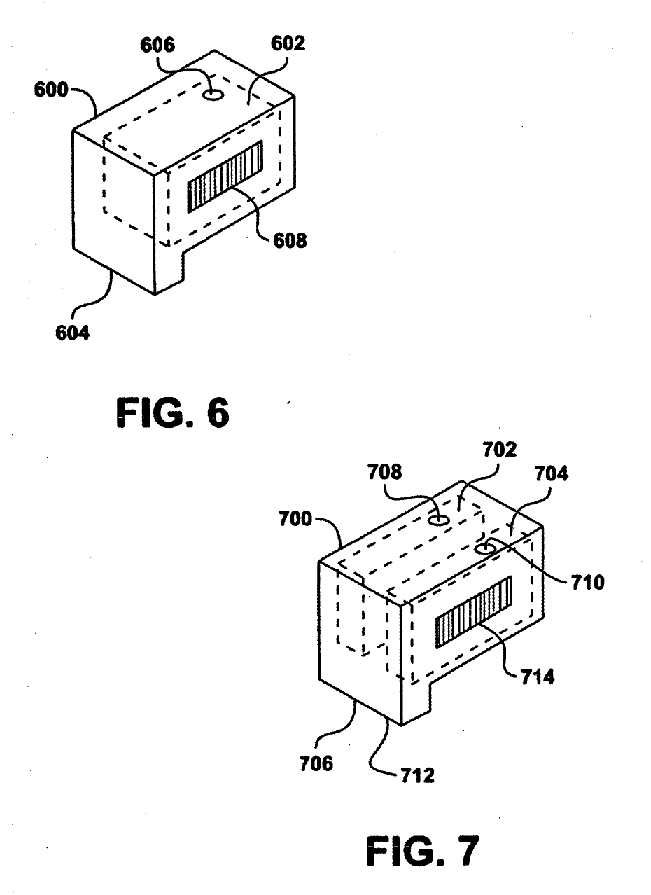

The treatment device can attach to a cartridge configured to store formulations, such as osteal functional coating formulations, sterilizing formulations, or texturing formulations. FIG. 6 illustrates an exemplary embodiment 600 of a cartridge for use in a treatment device. In an example, the cartridge can provide formulations for use by the treatment device. For example, the cartridge 600 can detachably couple to a treatment device. The cartridge 600 includes a container 602 and a dispensing nozzle 604. The cartridge 600 also can include a refill port 606 and can include a unique identifier 608.

The cartridge 600 is configured to store a formulation, such as a coating formulation, a sterilizing formulation, a texturing formulation, or any combination thereof. In an exemplary embodiment, coating matrix components and active agents can be combined together in a common compartment, such as container 602, in the cartridge body. The coating matrix components and the active agents are dispensed from a common nozzle, such as nozzle 604.

In an exemplary embodiment, the dispensing nozzle or orifice 604 is selectively controlled to dispense material. For example, the dispensing nozzle 604 can form a portion of a print head. As such, the nozzle 604 includes mechanisms for controlling the dispensing of a solution. Exemplary mechanisms include heater-driven bubble jet mechanisms, electrostatic mechanisms, and piezoelectric mechanisms. Alternatively, the orifice 604 provides material to a print head that is separate from the cartridge.

FIG. 7 illustrates an exemplary cartridge 700 that includes two or more containers 702 and 704. The cartridge 700 also includes one or more dispensing nozzles (706 and 712) and one or more refill ports (708 and 710). In an exemplary embodiment, the coating formulation components are separated from each other in dedicated compartments, such as containers 702 and 704. The compartments can be configured to dispense the coating formulation components through a common nozzle, such as nozzle 706. For example, the cartridge 700 can include dispensing structures configured to combine the coating formulation components prior to dispensing, such that the components are dispensed through one nozzle. In another example, the compartments can be configured to dispense the formulation through separate nozzles, such as nozzles 706 and 712.

in another exemplary embodiment, a container, such as the container 702, includes a solution having a first formulation and a second formulation can be stored in the container 704. For example, a coating formulation can be stored in the container 702 and a sterilizing formulation can be stored in the container 704. In another example, a coating formulation can be stored in the container 702 and a texturing formulation can be stored in the container 704. In a further example, a first coating formulation can be stored in the container 702 and a second coating formulation can be stored in the container 704. In another embodiment, the cartridge also can include a third formulation stored in a third container.

Alternatively, the second solution stored in container 704 can include a curing agent. For example, the curing agent can induce the components of a coating formulation to polymerize, crosslink or solidify. In another embodiment, the second solution can act as a diluent.

The one or more refill ports (708 and 710) can be used by a consumer, a service provider, or a manufacturer to refill the cartridge 700. In an exemplary embodiment, a medical professional can specify to a service provider or manufacturer the coating formulation with which the cartridge should be filled. For example, the consumer can enter the unique identifier 714 into a website and specify the desired coating formulation with which the cartridge 700 associated with the unique identifier 714 should be filled. The consumer can send the cartridge 700 to the service provider or manufacturer for refill.

In another exemplary embodiment, cartridges are selectively coupled to the treatment device. For example, a cartridge storing one composition can be replaced with a cartridge storing a different composition to produce implantable devices with different characteristics.

In a particular example, a cartridge can be filled in accordance with a prescription or a treatment protocol. For example, FIG. 16 includes a flow diagram of a method 1600 for preparing a cartridge. In an example, a cartridge is received at a facility, as illustrated at 1602. For example, a support facility can receive the cartridge from a clinical facility. In another example, a pharmacy or lab portion of the clinical facility can receive the cartridge.

The cartridge can be filled with a formulation, such as a coating formulation, as illustrated at 1604. For example, the cartridge can be filled based on a desired configuration of the implantable device. In a particular example, the cartridge can be filled with a formulation that includes active agents prescribed by a physician.

Once the cartridge is filled, the cartridge can be transferred to the facility, such as to the clinical facility or from one section of the clinical facility to a second section of the clinical facility, as illustrated at 1606. In a particular example, the cartridge includes a tracking number by which the cartridge can be identified for use in preparing an implantable device as prescribed.

Particular embodiments of the treatment device can be configured to treat implantable devices from various manufacturers or having various configurations. In addition, the treatment device can be configured to coat the implantable device using different coatings depending on the treatment prescribed for the patient.

The above-disclosed subject matter is to be considered illustrative, and not restrictive, and the appended claims are intended to cover all such modifications, enhancements, and other embodiments, which fall within the true scope of the present invention. For example, it will be understood by the skilled practitioner that various method or process steps described herein can be performed non-sequentially, as dictated by circumstances encountered in the field. Thus, to the maximum extent allowed by law, the scope of the present invention is to be determined by the broadest permissible interpretation of the following claims and their equivalents, and shall not be restricted or limited by the foregoing detailed description.

1. A method of preparing an implantable device having osteal functionality, the method comprising:
receiving the implantable device into a chamber located at a clinical setting; and
coating the implantable device within the chamber with an osteal functional coating.

2. The method of claim 1, further comprising texturing the surface of the implantable device within the chamber.

3. The method of claim 2, wherein texturing includes abrading with abrasive particulate.

4. The method of claim 2, wherein texturing includes contacting a surface of the implantable device with a chemical etchant.

5. The method of claim 1, further comprising sterilizing the implantable device within the chamber.

6. The method of claim 5, wherein sterilizing includes sterilizing with steam.

7. The method of claim 5, wherein sterilizing includes autoclaving.

8. The method of claim 5, wherein sterilizing includes contacting the implantable device with an antiseptic solution.

9. The method of claim 5, wherein sterilizing includes irradiating.

10. The method of claim 1, wherein coating includes dip coating.

11. The method of claim 1, wherein coating includes spray coating.

12. The method of claim 1, further comprising cleaning the implantable device within the chamber.

13. The method of claim 1, wherein the osteal functional coating includes an osteoconductive substance.

14. The method of claim 1, wherein the osteal functional coating includes an osteoinductive substance.

15. The method of claim 1, wherein the osteal functional coating includes a polymer.

16. The method of claim 1, wherein the clinical setting includes a surgical facility.

17. The method of claim 1, wherein the clinical setting includes a hospital.

18. (canceled)

19. A method of preparing an implantable device having osteal functionality, the method comprising:
receiving the implantable device into a chamber located at a surgical facility;
sterilizing the implantable device within the chamber; and
coating the implantable device within the chamber with an osteal functional coating.

20. (canceled)

21. (canceled)

22. (canceled)

23. (canceled)

24. (canceled)

25. (canceled)

26. The method of claim 19, further comprising texturing the surface of the implantable device within the chamber.

27. The method of claim 26, further comprising cleaning the implantable device within the chamber after texturing the surface of the implantable device.

28. A device comprising:
a sterilizing system configured to sterilize an implantable device; and
a coating system configured to apply an osteal functional coating to the implantable device.

29. The device of claim 28, further comprising a texturing system configured to texture a surface of the implantable device.

30. The device of claim 29, wherein the texturing system includes a fluid system to abrade the surface with an abrasive slurry.

31. The device of claim 29, wherein the texturing system includes a pneumatic system configured to direct a high velocity particulate at the surface.

32. The device of claim 28, wherein the sterilizing system includes an autoclave system.

33. The device of claim 28, wherein the sterilizing system includes a steam injection system.

34. The device of claim 28, wherein the sterilizing system includes an irradiating system.

35. The device of claim 28, wherein the coating system includes a dip coating system.

36. The device of claim 28, wherein the coating system includes a spray coating system.

37-71. (canceled)
